(19) United States
(12) Patent Application Publication
(10) Pub. No.: US 2022/0378811 A1
(43) Pub. Date: Dec. 1, 2022

(54) VENETOCLAX DOSING REGIMENS FOR USE IN TREATING MYELODYSPLASTIC SYNDROMES IN COMBINATION WITH AZACITIDINE

(71) Applicant: ABBVIE INC., North Chicago, IL (US)

(72) Inventors: John HAYSLIP, Satellite Beach, FL (US); Steve H. KYE, Vernon Hills, IL (US); William B. AINSWORTH, Evanston, IL (US)

(73) Assignee: ABBVIE INC., North Chicago, IL (US)

(21) Appl. No.: 17/741,359

(22) Filed: May 10, 2022

Related U.S. Application Data
(60) Provisional application No. 63/201,749, filed on May 11, 2021.

(51) Int. Cl. A61K 31/706; A61K 31/635; A61P 35/00; A61K 9/00

(52) U.S. Cl. CPC A61K 31/706; A61K 31/635; A61P 35/00; A61K 9/0019

(57) ABSTRACT

The invention described herein relates to therapeutic dosing regimens comprising administering venetoclax in combination with azacitidine for treating myelodysplastic syndromes (MDS).

### CROSS-REFERENCE TO RELATED APPLICATIONS

This application claims the benefit of U.S. Provisional Application No. 63/201,749, filed May 11, 2021, the disclosure of which is incorporated by reference herein in its entirety.

### FIELD OF THE INVENTION

This invention relates to methods for treating myelodysplastic syndromes (MDS) in a human subject comprising administering to the subject venetoclax in combination with azacitidine.

### BACKGROUND OF THE INVENTION

Myelodysplastic Syndromes (MDS) represent a heterogeneous group of clonal hematopoietic stem cell disorders with significant morbidity and high mortality. These syndromes are characterized by ineffective hematopoiesis that manifest clinically as cytopenias and a variable rate of transformation to acute myeloid leukemia (secondary or sAML). Although about one-third of all MDS patients later develop AML, MDS are not considered to be an early form of AML. The primary reason for death in MDS patients is not because of AML transformation, but due to consequences of bone marrow failure, and in particular neutropenia leading to infections, including septic shock, or thrombocytopenia leading to bleeding.

Approximately half (45%) of MDS patients present with higher-risk MDS risk (International Prognostic Scoring System (IPSS) overall score >1.5) and have a median survival less than one year with best supportive care. The only curative treatment for higher-risk MDS is an allogeneic stem cell or bone marrow transplantation. However, not all patients are eligible for this intensive treatment approach. If bone marrow transplantation is not possible, patients are typically treated with hypomethylating agents such as azacitidine. Currently, azacitidine is the only drug shown to prolong survival in treatment-naïve higher-risk MDS, however, overall outcomes need to be improved.

Venetoclax is an oral small molecule inhibitor of B-cell lymphoma 2 (BCL-2) that rapidly induces multiple hallmarks of apoptotic cell death. Venetoclax is being investigated in clinical oncology studies as a monotherapy and in combination with a variety of compounds for the treatment of a number of hematologic malignancies, including chronic lymphocytic leukemia (CLL) and acute myeloid leukemia (AML). However, the dosing regimen used in the first clinical trial of venetoclax in MDS produced deleterious side effects in certain patients. For example, two subjects developed fatal sepsis in the setting of severe neutropenia. Thus, there exists in the art a need for dosing regimen for MDS patients experiencing certain side effects.

### BRIEF SUMMARY OF THE INVENTION

The present disclosure relates to methods for treating myelodysplastic syndromes in a human subject, and in some aspects, more specifically treatment-naive higher-risk myelodysplastic syndromes.

In certain embodiments, a method for treating myelodysplastic syndromes in a human subject is provided. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and has at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle. In some aspects, the daily dose of azacitidine is reduced according to the bone marrow cellularity, the absolute neutrophil count nadir decrease, the white blood cell count nadir decrease, and the platelet count nadir count decrease.

In certain embodiments, a method for treating myelodysplastic syndromes in a human subject is provided. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% but no less than 33% during a next 28 day dosing cycle. The human subject has a baseline absolute neutrophil count of <1.5×10⁹/L, a baseline white blood cell count of <3×10⁹/L, or a baseline platelet count of <75×10⁹/L. The human subject has no improvement in cell line differentiation and the human subject has at least one condition selected from the group consisting of: an absolute neutrophil count nadir of ≥50% reduction, a white blood cell count nadir of ≥50% reduction, and a platelet count nadir of ≥50% reduction. In some aspects, the daily dose of azacitidine is reduced according to the bone marrow cellularity.

In certain embodiments, a method for treating myelodysplastic syndromes in a human subject is provided. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L, and a platelet count nadir of ≤50×10⁹/L; the daily dose of azacitidine is reduced from 75 mg/m² to ≤67% but no less than 50% during a next 28 day dosing cycle.

In certain embodiments, a method for treating myelodysplastic syndromes in a human subject, comprising administering to the human subject a daily dose of 400 mg of venetoclax for 14 days in a 28 day dosing cycle, and a daily dose of azacitidine for 7 days in the 28 day dosing cycle; wherein the daily dose of azacitidine is reduced from 75 mg/m² to 50% during a next 28 day dosing cycle. The human subject has a baseline absolute neutrophil count of ≥1.5×10⁹/L, a baseline white blood cell count of ≥3×10⁹/L, or a baseline platelet count of ≥75×10⁹/L. The human subject also has at least one of a condition selected from the group consisting of: an absolute neutrophil count nadir of ≤1.0×10⁹/L, and a platelet count nadir of ≤50×10⁹/L.

In certain embodiments, a method for treating myelodysplastic syndromes in a human subject is provided. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine is reduced from 75 mg/m² to ≤50 mg/m² but no less than 36 mg/m² during a next 28 day dosing cycle. The human subject has a baseline absolute neutrophil count of ≥1.5×10⁹/L, or a baseline platelet count of ≥75×10⁹/L; and a previous response of complete remission, partial remission, or marrow complete remission at the beginning of a prior dosing cycle. The human subject has at least one of a condition selected from the group consisting of: an absolute neutrophil count nadir of ≤0.500×10⁹/L, a platelet count nadir of ≤50×10⁹/L; wherein the baseline platelet was >100×10⁹/L; and a platelet count nadir of <50%; wherein the baseline platelet was ≤100×10⁹/L.

### BRIEF DESCRIPTION OF THE FIGURES

FIG. 1 is a plot of the mean value of the absolute neutrophil count versus study day cycle. The number of observations is shown in Table 11.

FIG. 2 is a plot of the mean value of the platelet count versus study day cycle. The number of observations is shown in Table 11.

FIGS. 3A-3F are bar graphs of hematologic toxicity showing the number of patients with worsening common terminology criteria grade over baseline per cycle. FIG. 3A is anemia.

FIG. 3B is febrile neutropenia. FIG. 3C is leukopenia. FIG. 3D is neutropenia. FIG. 3E is thrombocytopenia. FIG. 3F is infections.

FIGS. 4A-4C are bar graphs of gastrointestinal toxicity showing the number of patients with worsening common terminology criteria grade over baseline per cycle. FIG. 4A is diarrhea. FIG. 4B is vomiting. FIG. 4C is nausea.

### DETAILED DESCRIPTION OF THE INVENTION

This present disclosure relates to methods for treating treatment-naive higher-risk myelodysplastic syndromes (MDS) in a human subject comprising administering to the subject venetoclax in combination with azacitidine.

Although venetoclax has been administered to patients with AML who had a prior history of MDS (sAML), herein is the first disclosure evaluating venetoclax in combination with azacitidine in subjects with MDS, more specifically, in those subjects with treatment-naive higher-risk MDS, in which the dosing regimen is modified to account for subjects who have developed hematological toxicities after beginning treatment. Apart from the toxicity-specific dosing modifications disclosed herein, other significant differences between dosing venetoclax in combination with azacitidine for MDS versus AML include reducing the duration of venetoclax dosing from 28 to 14 days in the 28 day cycle for MDS, as well the lack of any dosing ramp up for subjects with MDS.

In certain embodiments, a method for treating myelodysplastic syndromes in a human subject is provided. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle.

“Venetoclax” is 4-(4-{[2-(4-chlorophenyl)-4,4-dimethylcyclohex-1-en-1-yl]methyl}piperazin-1-yl)-N-({3nitro-4-[(tetrahydro-2H-pyran-4-ylmethyl)amino]phenyl}sulfonyl)-2-(1H-pyrrolo[2,3-b]pyridin5-yloxy)benzamide. Venetoclax is a selective Bcl-2 inhibitor approved for adult patients with CLL and adult patients with newly diagnosed AML who are 75 years or older, or who are ineligible for intensive induction chemotherapy.

Azacitidine is 4-amino-1-β-D-ribofuranosyl-s-triazin-2(1H)-one. Azacitidine is supplied in a sterile form for reconstitution as a suspension for subcutaneous injection or reconstitution as a solution with further dilution for intravenous infusion.

The term “AE” as used herein refers to adverse event.

The term “AML” as used herein refers to acute myeloid leukemia.

The term “ANC” as used herein refers to absolute neutrophil count.

The term “CLL” as used herein refers to chronic lymphocytic leukemia.

The term “CML” as used herein refers to chronic myeloid leukemia.

The term “CMML” as used herein refers to chronic myelomonocytic leukemia.

The term “CR” as used herein refers to complete remission.

The term “CTC” as used herein refers to common terminology criteria.

The term “ECOG” as used herein refers to Eastern Cooperative Oncology Group.

The term “G-CSF” as used herein refers to granulocyte colony-stimulating factor.

The term “HRQoL” as used herein refers to health-related quality of life.

The term “HMAs” as used herein refers to hypomethylating agents.

The term “HR-MDS” as used herein refers to higher risk myelodysplastic syndromes.

The term “IPSS” as used herein refers to International Prognostic Scoring System.

The term “IPSS-R” as used herein refers to Revised International Prognostic Scoring System.

The term “JMML” as used herein refers to juvenile myelomonocytic leukemia.

The term “mCR” as used herein refers to marrow complete remission.

The term “MDS” as used herein refers to myelodysplastic syndromes.

The term “MPN” as used herein refers to myeloproliferative neoplasm.

The term “OS” as used herein refers to overall survival.

The term “PR” as used herein refers to partial remission.

The term “RAEB” as used herein refers to refractory anemia with excess blasts.

The term “sAML” as used herein refers to secondary acute myeloid leukemia.

The term “SE1” as used herein refers to Safety Expansion Cohort 1.

The term “SE2” as used herein refers to Safety Expansion Cohort 2.

The term “TEAE” as used herein refers to treatment-emergent adverse events.

The term “tMDS” as used herein refers to treatment-related or therapy-related myelodysplastic syndromes.

No improvement in cell line differentiation refers to the lack of clear improvement in cell differentiation at the time of the next cycle. For example, the percentage of mature granulocytes is lower and the absolute neutrophil count is lower than at onset of the dosing cycle.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹ L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 75% during the next 28 day dosing cycle; wherein the human subject has a bone marrow cellularity of 30-60%; and wherein the human subject has at least one of a condition selected from the group consisting of: an absolute neutrophil count nadir decrease of ≥75% from the baseline absolute neutrophil count, a white blood cell count nadir decrease of ≥75% from the baseline white blood cell count; and a platelet count nadir decrease of >75% from the baseline platelet count. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 75% during the next 28 day dosing cycle; wherein the human subject has a bone marrow cellularity of 30-60%; wherein the human subject has at least one of a condition selected from the group consisting of: an absolute neutrophil count nadir decrease of ≥75% from the baseline absolute neutrophil count, a white blood cell count nadir decrease of ≥75% from the baseline white blood cell count; and a platelet count nadir decrease of >75% from the baseline platelet count; and wherein the human subject has prior to a start of the next 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹ L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 75% during the next 28 day dosing cycle; wherein the human subject has a bone marrow cellularity of 30-60%; wherein the human subject has at least one of a condition selected from the group consisting of: an absolute neutrophil count nadir decrease of ≥75% from the baseline absolute neutrophil count, a white blood cell count nadir decrease of ≥75% from the baseline white blood cell count; and a platelet count nadir decrease of >75% from the baseline platelet count; wherein the human subject has prior to a start of the next 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir; wherein the daily dose of 400 mg of venetoclax is reduced from 14 to 7 days in a subsequent 28 day dosing cycle; and wherein the human subject has prior to a start of the subsequent 28 day dosing cycle, at least one condition selected from the group consisting of: a subsequent absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 50% during the next 28 day dosing cycle; wherein the human subject has a bone marrow cellularity of 15 to <30%; and wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 50% during the next 28 day dosing cycle; wherein the human subject has a bone marrow cellularity of 15 to <30%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the human subject has prior to a start of the next 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 50% during the next 28 day dosing cycle; wherein the human subject has a bone marrow cellularity of 15 to <30%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; wherein the human subject has prior to a start of the next 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir; wherein the daily dose of 400 mg of venetoclax is reduced from 14 to 7 days in a subsequent 28 day dosing cycle; and wherein the human subject has prior to a start of the subsequent 28 day dosing cycle, at least one condition selected from the group consisting of: a subsequent absolute neutrophil count of ≤25% above the absolute neutrophil count nadir and relative to a difference of the baseline absolute neutrophil count and the absolute neutrophil count nadir, a subsequent white blood cell count of ≤25% above the white blood cell count nadir and relative to a difference of the baseline white blood cell count and the white blood cell count nadir, and a subsequent platelet count of ≤25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 50% during the next 28 day dosing cycle; wherein the human subject has a bone marrow cellularity of 15 to <30%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the human subject has at least one condition selected from the group consisting of: an absolute neutrophil count nadir decrease of >75% from the baseline absolute neutrophil count, a white blood cell count nadir decrease of >75% from the baseline white blood cell count, and a platelet count nadir decrease of >75% from the baseline platelet count. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of >50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 50% during the next 28 day dosing cycle; wherein the human subject has a bone marrow cellularity of 15 to <30%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the human subject has at least one condition selected from the group consisting of: an absolute neutrophil count nadir decrease of 50-75% from the baseline absolute neutrophil count, a white blood cell count nadir decrease of 50-75% from the baseline white blood cell, and a platelet count nadir decrease of 50-75% from the baseline platelet count. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of >50% reduction, a white blood cell count nadir of >50% reduction, and a platelet count nadir of >50% reduction; the daily dose of azacitidine of 75 mg/m² is reduced to <75% during but no less than 33% a next 28 day dosing cycle; wherein the daily dose of azacitidine of 75 mg/m² is reduced to 33% during the next 28 day dosing cycle; wherein the human subject has a bone marrow cellularity <15%; and wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of >50% reduction, a white blood cell count nadir of >50% reduction, and a platelet count nadir of >50% reduction; the daily dose of azacitidine of 75 mg/m² is reduced to <75% but no less than 33% during a next 28 day dosing cycle; wherein the daily dose of azacitidine of 75 mg/m² is reduced to 33% during the next 28 day dosing cycle; wherein the human subject has a bone marrow cellularity <15%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the human subject has prior to a start of the next 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naive higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of >50% reduction, and a platelet count nadir of ≥50% reduction; the daily dose of azacitidine of 75 mg/m² is reduced to ≤75% during but no less than 33% a next 28 day dosing cycle; wherein the daily dose of azacitidine of 75 mg/m² is reduced to 33% during the next 28 day dosing cycle; wherein the human subject has a bone marrow cellularity <15%; wherein the human subject has at least one of a condition selected from the group consisting of: an absolute neutrophil count nadir decrease of >50% from the baseline absolute neutrophil count, a white blood cell count nadir decrease of >50% from the baseline white blood cell count, and a platelet count nadir decrease of >50% from the baseline platelet count; wherein the human subject has prior to a start of the next 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir; wherein the daily dose of 400 mg of venetoclax is reduced from 14 to 7 days in a subsequent 28 day dosing cycle; and wherein the human subject has prior to a start of the subsequent 28 day dosing cycle, at least one condition selected from the group consisting of: a next subsequent neutrophil count of >25% above the absolute neutrophil count nadir and relative to a difference of the baseline absolute neutrophil count and the absolute neutrophil count nadir, a subsequent white blood cell count of >25% above the white blood cell count nadir and relative to a difference of the baseline white blood cell count and the white blood cell count nadir, and a subsequent platelet count of >25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 33% during the next 28 day dosing cycle; wherein the human subject has a bone marrow cellularity <15%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the daily dose of 400 mg of venetoclax is reduced from 14 to 7 days in a subsequent 28 day dosing cycle; and wherein the human subject has prior to a start of the subsequent 28 day dosing cycle, at least one condition selected from the group consisting of: a subsequent neutrophil count of >25% above the absolute neutrophil count nadir and relative to a difference of the baseline absolute neutrophil count and the absolute neutrophil count nadir, a subsequent white blood cell count of >25% above the white blood cell count nadir and relative to a difference of the baseline white blood cell count and the white blood cell count nadir, and a subsequent platelet count of >25% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of >1 day; and further comprises a delay prior to the subsequent 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≥75% during but no less than 33% a next 28 day dosing cycle; wherein the daily dose of azacitidine of 75 mg/m² is reduced to 33% during the next 28 day dosing cycle; wherein the human subject has a bone marrow cellularity <15%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the human subject has at least one condition selected from the group consisting of: an absolute neutrophil count nadir decrease of >75% from the baseline absolute neutrophil count, a white blood cell count nadir decrease of >75% from the baseline white blood cell count, and a platelet count decrease of >75% from the baseline platelet count. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of <1.5×10⁹/L, a baseline white blood cell count of <3×10⁹/L, or a baseline platelet count of <75×10⁹/L; no improvement in cell line differentiation; and at least one of a condition selected from the group consisting of: an absolute neutrophil count nadir of ≥50% reduction, a white blood cell count nadir of ≥50% reduction, and a platelet count nadir of ≥50% reduction; the daily dose of azacitidine of 75 mg/m² is reduced to ≤75% but no less than 33% during a next 28 day dosing cycle; wherein the daily dose of azacitidine of 75 mg/m² is reduced to 33% during the next 28 day dosing cycle; wherein the human subject has a bone marrow cellularity <15%; wherein the human subject has at least one of a condition selected from the group consisting of: an absolute neutrophil count nadir decrease of ≥50% from the baseline absolute neutrophil count, a white blood cell count nadir decrease of ≥50% from the baseline white blood cell count, and a platelet count nadir decrease of ≥50% from the baseline platelet count; and wherein the human subject has at least one condition selected from the group consisting of: an absolute neutrophil count nadir decrease of 50-75% from the baseline absolute neutrophil count, a white blood cell count nadir decrease of 50-75% from the baseline white blood cell count relative to the white blood cell count nadir, and a platelet count nadir decrease of 50-75% from the baseline platelet count. In some aspects, the azacitidine is administered intravenously. In other aspects, the azacitidine is administered subcutaneously.

In certain embodiments, a method for treating myelodysplastic syndromes in a human subject is provided. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% during but no less than 33% a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; and wherein the human subject has at least one condition selected from the group consisting of: an absolute neutrophil count nadir of ≥50% reduction, a white blood cell count nadir of ≥50% reduction, and a platelet count nadir of ≥50% reduction.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; and wherein the human subject has at least one condition selected from the group consisting of: an absolute neutrophil count nadir of ≥50% reduction, a white blood cell count nadir of ≥50% reduction, and a platelet count nadir of ≥50% reduction.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; wherein the human subject has at least one condition selected from the group consisting of: an absolute neutrophil count nadir of ≥50% reduction, a white blood cell count nadir of ≥50% reduction, and a platelet count nadir of ≥50% reduction; wherein the daily dose of azacitidine of 75 mg/m² is reduced to 50% during the next 28 day dosing cycle; and wherein the human subject has a bone marrow cellularity of 15-50%. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; wherein the human subject has at least one condition selected from the group consisting of: an absolute neutrophil count nadir of ≥50% reduction, a white blood cell count nadir of ≥50% reduction, and a platelet count nadir of ≥50% reduction; wherein the daily dose of azacitidine of 75 mg/m² is reduced to 50% but no less than 33% during the next 28 day dosing cycle; wherein the human subject has a bone marrow cellularity of 15-50%; and wherein the human subject has prior to a start of the next 28 day dosing cycle, at least one condition selected from the group consisting of: a next absolute neutrophil count increase of <50% above the absolute neutrophil count nadir and relative to a difference of the baseline absolute neutrophil count and the absolute neutrophil count nadir, a next white blood cell count increase of <50% above the white blood cell count nadir and relative to a difference of the baseline white blood cell count and the white blood cell count nadir, and a next platelet count increase of <50% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; wherein the human subject has at least one condition selected from the group consisting of: an absolute neutrophil count nadir of ≥50% reduction, a white blood cell count nadir of ≥50% reduction, and a platelet count nadir of ≥50% reduction; wherein the daily dose of azacitidine of 75 mg/m² is reduced to 50% during the next 28 day dosing cycle; wherein the human subject has a bone marrow cellularity of 15-50%;

wherein the human subject has prior to a start of the next 28 day dosing cycle, at least one condition selected from the group consisting of: a next absolute neutrophil count increase of <50% above the absolute neutrophil count nadir and relative to a difference of the baseline absolute neutrophil count and the absolute neutrophil count nadir, a next white blood cell count increase of <50% above the white blood cell count nadir and relative to a difference of the baseline white blood cell count and the white blood cell count nadir, and a next platelet count increase of <50% above the platelet count nadir and relative to a difference of the baseline platelet count and the platelet count nadir; and wherein the daily dose of 400 mg of venetoclax is reduced from 14 to 7 days in a subsequent 28 day dosing cycle; wherein the human subject has prior to a start of the subsequent 28 day dosing cycle, at least one condition selected from the group consisting of: a subsequent absolute neutrophil count of <50% above the absolute neutrophil count nadir and relative to a difference of the baseline absolute neutrophil count and the absolute neutrophil count nadir, a subsequent white blood cell count of <50% above the white blood cell count nadir and relative to a difference of the baseline white blood cell count and the white blood cell count nadir, and a subsequent platelet count of <50% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of >1 day; and further comprises a delay prior to the subsequent 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to≤50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; wherein the human subject has at least one condition selected from the group consisting of: an absolute neutrophil count nadir of ≥50% reduction, a white blood cell count nadir of ≥50% reduction, and a platelet count nadir of ≥50% reduction; wherein the daily dose of azacitidine of 75 mg/m² is reduced to 33% during the next 28 day dosing cycle; and wherein the human subject has a bone marrow cellularity of <15%. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to <50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; wherein the human subject has at least one condition selected from the group consisting of: an absolute neutrophil count nadir of ≥50% reduction, a white blood cell count nadir of ≥50% reduction, and a platelet count nadir of ≥50% reduction; wherein the daily dose of azacitidine of 75 mg/m² is reduced to 33% during the next 28 day dosing cycle; and wherein the human subject has a bone marrow cellularity of <15%; wherein the human subject has prior to a start of the next 28 day dosing cycle, at least one condition selected from the group consisting of: a next absolute neutrophil count increase of <50% above the absolute neutrophil count nadir and relative to a difference of the baseline absolute neutrophil count and the absolute neutrophil count nadir, a next white blood cell count increase of <50% above the white blood cell count nadir and relative to a difference of the baseline white blood cell count and the white blood cell count nadir; and a next platelet count increase of <50% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of azacitidine for 7 days in the 28 day dosing cycle; wherein the daily dose of azacitidine of 75 mg/m² is reduced to ≤50% but no less than 33% during a next 28 day dosing cycle; wherein the human subject has a baseline absolute neutrophil count of <1.5×10⁹/L, a baseline white blood cell count of <3×10⁹/L, or a baseline platelet count of <75×10⁹/L; wherein the human subject has no improvement in cell line differentiation; wherein the human subject has at least one condition selected from the group consisting of: an absolute neutrophil count nadir of ≥50% reduction, a white blood cell count nadir of ≥50% reduction, and a platelet count nadir of ≥50% reduction; wherein the daily dose of azacitidine of 75 mg/m² is reduced to 33% during the next 28 day dosing cycle; wherein the human subject has a bone marrow cellularity of <15%; wherein the human subject has prior to a start of the next 28 day dosing cycle, at least one condition selected from the group consisting of: a next absolute neutrophil count increase of <50% above the absolute neutrophil count nadir and relative to a difference of the baseline absolute neutrophil count and the absolute neutrophil count nadir, a next white blood cell count increase of <50% above the white blood cell count nadir and relative to a difference of the baseline white blood cell count and the white blood cell count nadir; and a next platelet count increase of <50% above the platelet count nadir and relative to a difference of the baseline platelet count and the platelet count nadir; and wherein the daily dose of 400 mg of venetoclax is reduced from 14 to 7 days in a subsequent 28 day dosing cycle; wherein the human subject has prior to a start of the subsequent 28 day dosing cycle, at least one condition selected from the group consisting of: a subsequent absolute neutrophil count of <50% above the absolute neutrophil count nadir and relative to a difference of the baseline absolute neutrophil count and the absolute neutrophil count nadir, a subsequent white blood cell count of <50% above the white blood cell count nadir and relative to a difference of the baseline white blood cell count and the white blood cell count nadir, and a subsequent platelet count of <50% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has: a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to ≤67% but no less than 50% during a next 28 day dosing cycle.

In certain embodiments, a method for treating myelodysplastic syndromes in a human subject is provided, wherein the myelodysplastic syndromes are treatment-naive higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has: a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to ≤67% but no less than 50% during a next 28 day dosing cycle.

In certain embodiments, a method for treating myelodysplastic syndromes in a human subject is provided, wherein the myelodysplastic syndromes are treatment-naive higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to <67% but no less than 50% during a next 28 day dosing cycle; wherein the daily dose of azacitidine is reduced from 75 mg/m² to 67%; and wherein the human subject has at least one condition selected from the group consisting of: the absolute neutrophil count nadir is from 0.5×10⁹/L to 1.5×10⁹/L;

and the platelet count nadir is from 25×10⁹/L to 50×10⁹/L. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to ≤67% but no less than 50% during a next 28 day dosing cycle; wherein the daily dose of azacitidine is reduced from 75 mg/m² to 67%; wherein the human subject has at least one condition selected from the group consisting of: the absolute neutrophil count nadir is from 0.5×10⁹/L to 1.5×10⁹/L; and the platelet count nadir is from 25×10⁹/L to 50×10⁹/L; and wherein the human subject has prior to a start of the next 28 day dosing cycle, at least one condition selected from the group consisting of: a next absolute neutrophil count of ≤1.5×10⁹/L; and a next platelet count of ≤50×10⁹/L. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to ≤67% but no less than 50% during a next 28 day dosing cycle; wherein the daily dose of azacitidine is reduced from 75 mg/m² to 67%; wherein the human subject has at least one condition selected from the group consisting of: the absolute neutrophil count nadir is from 0.5×10⁹/L to 1.5×10⁹/L; and the platelet count nadir is from 25×10⁹/L to 50×10⁹/L; wherein the human subject has prior to a start of the next 28 day dosing cycle, at least one condition selected from the group consisting of: a next absolute neutrophil count of ≤1.5×10⁹/L; and a next platelet count of ≤50×10⁹/L; wherein the daily dose of 400 mg of venetoclax is reduced from 14 to 7 days in a subsequent 28 day dosing cycle; and wherein the human subject has prior to a start of the subsequent 28 day dosing cycle at least one condition selected from the group consisting of: a subsequent absolute neutrophil count of ≤1.5×10⁹/L; and a subsequent platelet count of ≤50×10⁹/L. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to ≤67% but no less than 50% during a next 28 day dosing cycle; wherein the daily dose of azacitidine is reduced from 75 mg/m² to 50%; and wherein the human subject has at least one condition selected from the group consisting of: the absolute neutrophil count nadir is <0.5×10⁹/L; and the platelet count nadir is <25×10⁹/L. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to <67% but no less than 50% during a next 28 day dosing cycle; wherein the daily dose of azacitidine is reduced from 75 mg/m² to 50%; wherein the human subject has at least one condition selected from the group consisting of: the absolute neutrophil count nadir is <0.5×10⁹/L; and the platelet count nadir is <25×10⁹/L; and wherein the human subject has prior to a start of the next 28 day dosing cycle, at least one condition selected from the group consisting of: a next absolute neutrophil count of ≤1.5×10⁹/L; and a next platelet count of ≤50×10⁹/L. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein if the human subject has a baseline absolute neutrophil count of ≥1.5×10⁹/L, a baseline white blood cell count of ≥3×10⁹/L, or a baseline platelet count of >75×10⁹/L; and at least one of a condition selected from the group consisting of: an absolute neutrophil count nadir of ≤1.5×10⁹/L; and a platelet count nadir of ≤50×10⁹/L; the daily dose of azacitidine is reduced from 75 mg/m² to ≤67% but no less than 50% during a next 28 day dosing cycle; wherein the daily dose of azacitidine is reduced from 75 mg/m² to 50%; wherein the human subject has at least one condition selected from the group consisting of: the absolute neutrophil count nadir is <0.5×10⁹/L; and the platelet count nadir is <25×10⁹/L; wherein the human subject has prior to a start of the next 28 day dosing cycle, at least one condition selected from the group consisting of: a next absolute neutrophil count of ≤1.5×10⁹/L; and a next platelet count of ≤50×10⁹/L; wherein the daily dose of 400 mg of venetoclax is reduced from 14 to 7 days in a subsequent 28 day dosing cycle; and wherein the human subject has prior to a start of the subsequent 28 day dosing cycle at least one condition selected from the group consisting of: a subsequent absolute neutrophil count of ≤1.5×10⁹/L; and a subsequent platelet count of ≤50×10⁹/L. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to 50% during a next 28 day dosing cycle; wherein the human subject has a baseline absolute neutrophil count of ≥1.5×10⁹/L, a baseline white blood cell count of ≥3 ×10⁹/L, or a baseline platelet count of ≥75×10⁹/L; and wherein the human subject has at least one of a condition selected from the group consisting of: an absolute neutrophil count nadir of <1.0×10⁹/L; and a platelet count nadir of ≤50×10⁹/L.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to 50% during a next 28 day dosing cycle; wherein the human subject has a baseline absolute neutrophil count of ≥1.5×10⁹/L, a baseline white blood cell count of ≥3 ×10⁹/L, or a baseline platelet count of ≥75×10⁹/L; and wherein the human subject has at least one of a condition selected from the group consisting of: an absolute neutrophil count nadir of ≤1.0×10⁹/L; and a platelet count nadir of ≤50×10⁹/L. In some aspects, the azacitidine is administered intravenously. In other aspects, the azacitidine is administered subcutaneously.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to 50% during a next 28 day dosing cycle; wherein the human subject has a baseline absolute neutrophil count of ≥1.5×10⁹/L, a baseline white blood cell count of ≥3×10⁹/L, or a baseline platelet count of ≥75×10⁹/L; wherein the human subject has at least one of a condition selected from the group consisting of: an absolute neutrophil count nadir of ≤1.0×10⁹/L; and a platelet count nadir of ≤50×10⁹/L; and wherein the human subject has prior to a start of the next 28 day dosing cycle, at least one condition selected from the group consisting of: a subsequent absolute neutrophil count increase of <50% above the absolute neutrophil count nadir and relative to a difference of the baseline absolute neutrophil count and the absolute neutrophil count nadir; and a subsequent platelet count increase of <50% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the next 28 day dosing cycle of ≥1 day.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to 50% during a next 28 day dosing cycle; wherein the human subject has a baseline absolute neutrophil count of ≥1.5×10⁹/L, a baseline white blood cell count of ≥3×10⁹/L, or a baseline platelet count of ≥75×10⁹/L; wherein the human subject has at least one of a condition selected from the group consisting of: an absolute neutrophil count nadir of ≤1.0×10⁹/L; and a platelet count nadir of ≤50×10⁹/L; wherein the human subject has prior to a start of the next 28 day dosing cycle, at least one condition selected from the group consisting of: a subsequent absolute neutrophil count increase of <50% above the absolute neutrophil count nadir and relative to a difference of the baseline absolute neutrophil count and the absolute neutrophil count nadir; and a subsequent platelet count increase of <50% above the platelet count nadir and relative to a difference of the baseline platelet count and the platelet count nadir; wherein the daily dose of 400 mg of venetoclax is reduced from 14 to 7 days in a subsequent 28 day dosing cycle; and wherein the human subject has prior to the start of the subsequent 28 day dosing cycle, at least one condition selected from the group consisting of: a subsequent absolute neutrophil count increase of <50% above the absolute neutrophil count nadir and relative to a difference of the baseline absolute neutrophil count and the absolute neutrophil count nadir; and a subsequent platelet count increase of <50% above the platelet count nadir and relative to a difference of the baseline platelet count and the platelet count nadir. In some aspects, the azacitidine is administered intravenously. In other aspects, the azacitidine is administered subcutaneously. In yet other aspects, the method further comprises a delay prior to the subsequent 28 day dosing cycle of ≥1 day. In still other aspects, the method further comprises a delay prior to the next 28 day dosing cycle of ≥1 day; and further comprises a delay prior to the subsequent 28 day dosing cycle of ≥1 day.

In certain embodiments, a method for treating myelodysplastic syndromes in a human subject is provided. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to ≤50 mg/m² but no less than 36 mg/m² during a next 28 day dosing cycle; wherein the human subject has a baseline absolute neutrophil count of ≥1.5×10⁹/L, or a baseline platelet count of ≥75×10⁹/L; wherein the human subject has a previous response of complete remission, partial remission, or marrow complete remission at the beginning of a prior dosing cycle; and wherein the human subject has at least one of a condition selected from the group consisting of: an absolute neutrophil count nadir of ≤0.500×10⁹/L, a platelet count nadir of ≤50×10⁹/L; wherein the baseline platelet was >100×10⁹/L, and a platelet count nadir of <50%; wherein the baseline platelet was ≤100×10⁹/L.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to ≤50 mg/m² but no less than 36 mg/m² during a next 28 day dosing cycle; wherein the human subject has a baseline absolute neutrophil count of ≥1.5×10⁹/L, or a baseline platelet count of ≥75×10⁹/L; wherein the human subject has a previous response of complete remission, partial remission, or marrow complete remission at the beginning of a prior dosing cycle; and wherein the human subject has at least one of a condition selected from the group consisting of: an absolute neutrophil count nadir of ≤0.500×10⁹/L, and a platelet count nadir of ≤50×10⁹/L; wherein the baseline platelet was >100×10⁹/L, a platelet count nadir of <50%; wherein the baseline platelet was ≤100×10⁹/L.

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to <50 mg/m² but no less than 36 mg/m² during a next 28 day dosing cycle; wherein the human subject has a baseline absolute neutrophil count of ≥1.5×10⁹/L, or a baseline platelet count of ≥75×10⁹/L; wherein the human subject has a previous response of complete remission, partial remission, or marrow complete remission at the beginning of a prior dosing cycle; wherein the human subject has at least one of a condition selected from the group consisting of: an absolute neutrophil count nadir of ≤0.500×10⁹/L, and a platelet count nadir of ≤50×10⁹/L; wherein the baseline platelet was >100×10⁹/L, a platelet count nadir of <50%; wherein the baseline platelet was ≤100×10⁹/L; and wherein the daily dose of azacitidine is reduced from 75 mg/m² to 50 mg/m².

In certain embodiments, a method for treating myelodysplastic syndromes in a human subject is provided, wherein the myelodysplastic syndromes are treatment-naïve higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to <50 mg/m² but no less than 36 mg/m² during a next 28 day dosing cycle; wherein the human subject has a baseline absolute neutrophil count of ≥1.5×10⁹/L, or a baseline platelet count of ≥75×10⁹/L; wherein the human subject has a previous response of complete remission, partial remission, or marrow complete remission at the beginning of a prior dosing cycle; wherein the human subject has at least one of a condition selected from the group consisting of: an absolute neutrophil count nadir of ≤0.500×10⁹/L, and a platelet count nadir of ≤50×10⁹/L; wherein the baseline platelet was >100×10⁹/L, a platelet count nadir of <50%; wherein the baseline platelet was ≤100×10⁹/L; wherein the daily dose of azacitidine is reduced from 75 mg/m² to 50 mg/m²; and wherein the daily dose of azacitidine is reduced from 50 mg/m² to 36 mg/m² in a successive 28 day dosing cycle.

In certain embodiments, a method for treating myelodysplastic syndromes in a human subject is provided, wherein the myelodysplastic syndromes are treatment-naive higher-risk myelodysplastic syndromes. The method comprises administering to the human subject a daily dose of 400 mg of venetoclax for 14 days in a 28 day dosing cycle, and a daily dose of 75 mg/m² of azacitidine for 7 days in the 28 day dosing cycle; wherein the daily dose of azacitidine is reduced from 75 mg/m² to <50 mg/m² but no less than 36 mg/m² during a next 28 day dosing cycle; wherein the human subject has a baseline absolute neutrophil count of ≥1.5×10⁹/L, or a baseline platelet count of ≥75×10⁹/L; wherein the human subject has a previous response of complete remission, partial remission, or marrow complete remission at the beginning of a prior dosing cycle; wherein the human subject has at least one of a condition selected from the group consisting of: an absolute neutrophil count nadir of ≤0.500×10⁹/L, and a platelet count nadir of ≤50×10⁹/L; wherein the baseline platelet was >100×10⁹/L, a platelet count nadir of <50%; wherein the baseline platelet was ≤100×10⁹/L; wherein the daily dose of azacitidine is reduced from 75 mg/m² to 50 mg/m²; wherein the daily dose of azacitidine is reduced from 50 mg/m² to 36 mg/m² in a successive 28 day dosing cycle; and wherein the daily dose of 400 mg of venetoclax is reduced from 14 to 7 days in a subsequent 28 day dosing cycle.

In certain embodiments, a method for treating myelodysplastic syndromes in a human subject is provided. The method comprises (a) obtaining a baseline absolute neutrophil count, a baseline white blood cell count, and a baseline platelet count from the human subject, (b) treating the human subject with an initial dose of venetoclax and an initial dose of azacitidine during a first treatment cycle, (c) comparing the baseline absolute neutrophil count, baseline white blood cell count, and baseline platelet count with a subsequent absolute neutrophil count, subsequent white blood cell count, and subsequent platelet count obtained after the first treatment cycle, and (d) if the subsequent absolute neutrophil count nadir is ≥50% of the baseline neutrophil count, the subsequent white blood cell count nadir is ≥50% of the baseline neutrophil count, or the subsequent platelet count nadir is ≥50% of the baseline platelet count, treating the human subject with a dose of azacitidine that is ≤75% but no less than 33% of the initial dose of azacitidine in a subsequent treatment cycle. In some aspects, the myelodysplastic syndromes are treatment-naive higher-risk myelodysplastic syndromes. In other aspects, the initial dose of venetoclax is 400 mg and the initial dose of azacitidine is 75 mg/m². In yet other aspects, the dosing cycle is 28 days. In yet other aspects, the human subject is administered a daily dose of venetoclax for 14 days in a 28 day dosing cycle and a daily dose of 75 mg/m² of azacitidine for 7 days in the 28 day dosing cycle. In yet other aspects, the daily dose of 400 mg of venetoclax is reduced from 14 to 7 days in a further subsequent 28 day dosing cycle if the human subject has prior to a start of the further subsequent 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In yet other aspects, the azacitidine is administered intravenously. In yet other aspects, the azacitidine is administered subcutaneously.

In certain embodiments, a method for treating myelodysplastic syndromes in a human subject is provided. The method comprises (a) obtaining a baseline absolute neutrophil count, a baseline white blood cell count, and a baseline platelet count from the human subject, (b) treating the human subject with an initial dose of venetoclax and an initial dose of azacitidine during a first treatment cycle, (c) comparing the baseline absolute neutrophil count, baseline white blood cell count, and baseline platelet count with a subsequent absolute neutrophil count, subsequent white blood cell count, and subsequent platelet count obtained after the first treatment cycle, and (d) if the subsequent absolute neutrophil count nadir is ≥50% of the baseline neutrophil count, the subsequent white blood cell count nadir is ≥50% of the baseline neutrophil count, or the subsequent platelet count nadir is ≥50% of the baseline platelet count, treating the human subject with a dose of azacitidine that is ≤50% but no less than 33% of the initial dose of azacitidine in a subsequent treatment cycle. In some aspects, the myelodysplastic syndromes are treatment-naive higher-risk myelodysplastic syndromes. In other aspects, the initial dose of venetoclax is 400 mg and the initial dose of azacitidine is 75 mg/m². In yet other aspects, the dosing cycle is 28 days. In yet other aspects, the human subject is administered a daily dose of venetoclax for 14 days in a 28 day dosing cycle and a daily dose of 75 mg/m² of azacitidine for 7 days in the 28 day dosing cycle. In yet other aspects, the daily dose of 400 mg of venetoclax is reduced from 14 to 7 days in a further subsequent 28 day dosing cycle if the human subject has prior to a start of the further subsequent 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In yet other aspects, the azacitidine is administered intravenously. In yet other aspects, the azacitidine is administered subcutaneously.

In certain embodiments, a method for treating myelodysplastic syndromes in a human subject is provided. The method comprises (a) obtaining a baseline absolute neutrophil count, a baseline white blood cell count, and a baseline platelet count from the human subject, (b) treating the human subject with an initial dose of venetoclax and an initial dose of azacitidine during a first treatment cycle, (c) comparing the baseline absolute neutrophil count, baseline white blood cell count, and baseline platelet count with a subsequent absolute neutrophil count, subsequent white blood cell count, and subsequent platelet count obtained after the first treatment cycle, and (d) if the human subject has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of ≤1.5×10⁹/L, and a platelet count nadir of ≤50×10⁹/L; the dose of azacitidine is reduced from 75 mg/m² to ≤67% but no less than 50% during a next 28 day dosing cycle. In some aspects, the myelodysplastic syndromes are treatment-naïve higher-risk myelodysplastic syndromes. In other aspects, the initial dose of venetoclax is 400 mg and the initial dose of azacitidine is 75 mg/m². In yet other aspects, the dosing cycle is 28 days. In yet other aspects, the human subject is administered a daily dose of venetoclax for 14 days in a 28 day dosing cycle and a daily dose of 75 mg/m² of azacitidine for 7 days in the 28 day dosing cycle. In yet other aspects, the daily dose of 400 mg of venetoclax is reduced from 14 to 7 days in a further subsequent 28 day dosing cycle if the human subject has prior to a start of the further subsequent 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In yet other aspects, the azacitidine is administered intravenously. In yet other aspects, the azacitidine is administered subcutaneously.

In certain embodiments, a method for treating myelodysplastic syndromes in a human subject is provided. The method comprises (a) obtaining a baseline absolute neutrophil count, a baseline white blood cell count, and a baseline platelet count from the human subject, (b) treating the human subject with an initial dose of venetoclax and an initial dose of azacitidine during a first treatment cycle, (c) comparing the baseline absolute neutrophil count, baseline white blood cell count, and baseline platelet count with a subsequent absolute neutrophil count, subsequent white blood cell count, and subsequent platelet count obtained after the first treatment cycle, and (d) has a baseline absolute neutrophil count of ≥1.5×10⁹/L, a baseline white blood cell count of ≥3×10⁹/L, or a baseline platelet count of ≥75×10⁹/L; and at least one of a condition selected from the group consisting of: an absolute neutrophil count nadir of <1.0×10⁹/L, and a platelet count nadir of ≤50×10⁹/L, the dose of azacitidine is reduced from 75 mg/m² to 50% during a next 28 day dosing cycle. In some aspects, the myelodysplastic syndromes are treatment-naive higher-risk myelodysplastic syndromes. In other aspects, the initial dose of venetoclax is 400 mg and the initial dose of azacitidine is 75 mg/m². In yet other aspects, the dosing cycle is 28 days. In yet other aspects, the human subject is administered a daily dose of venetoclax for 14 days in a 28 day dosing cycle and a daily dose of 75 mg/m² of azacitidine for 7 days in the 28 day dosing cycle. In yet other aspects, the daily dose of 400 mg of venetoclax is reduced from 14 to 7 days in a further subsequent 28 day dosing cycle if the human subject has prior to a start of the further subsequent 28 day dosing cycle, at least one condition selected from the group consisting of: a next absolute neutrophil count of ≤25% above the absolute neutrophil count nadir and relative to a difference of the baseline absolute neutrophil count and the absolute neutrophil count nadir, a next white blood cell count of ≤25% above the white blood cell count nadir and relative to a difference of the baseline white blood cell count and the white blood cell count nadir, and a next platelet count of ≤25% above the platelet count nadir and relative to a difference of the baseline platelet count and the platelet count nadir. In yet other aspects, the azacitidine is administered intravenously. In yet other aspects, the azacitidine is administered subcutaneously.

### EXAMPLES

In order that the invention described herein may be more fully understood, the following examples are set forth.

A multicenter, non-randomized Phase 1b study in adults with previously untreated higher-risk MDS, defined as IPSS risk categories of Int-2 or High (IPSS overall score ≥1.5) was initiated. The original protocol randomized patients into 1 of 3 treatment groups: venetoclax 800 mg +azacitidine, venetoclax 400 mg +azacitidine, and azacitidine monotherapy. Under the original protocol, venetoclax was administered on Days 1 through 28 of each 28-day-cycle and dosing was initiated according to a ramp-up dosing schedule in Cycle 1. With this dosing schedule, 2 patients developed fatal sepsis in the setting of severe neutropenia, after which the study was placed on partial clinical hold and enrollment was suspended. The partial clinical hold was lifted based on a revised protocol, which ultimately resulted in a lower incidence of infections and leukopenia events.

### STUDY DESIGN

### Subject Inclusion Criteria

Subjects 18 years or older diagnosed with treatment-naïve IPSS intermediate-2 or high-risk myelodysplastic syndromes with ECOG ≤2 were enrolled. For this study, inclusion criteria included the following:

1. Subject must be ≥18 years of age.

2. Subject must have documented diagnosis of previously untreated de novo MDS with:
a. International Prognostic Scoring System (IPSS) risk categories Intermediate-2 or High (i.e., minimum IPSS score of 1.5) OR Revised IPSS (IPSS-R) categories Intermediate, High or Very High (score of >3); and
b. Presence of <20% bone marrow blasts per bone marrow biopsy/aspirate.

3. Subject must have an Eastern Cooperative Oncology Group (ECOG) performance score of ≤2.

According to the International Prognostic Scoring System, myelodysplastic syndromes patients are grouped into two major risk groups, low risk and higher-risk. Higher-risk myelodysplastic syndromes as used herein are defined as an International Prognostic Scoring System (IPSS) risk categories Int-2 or High (i.e., minimum IPSS score of 1.5) or Revised IPSS (IPSS-R) categories Intermediate, High or Very High (overall score of >3).

TABLE 1
International Prognostic Scoring System (IPSS)

| | Score | | | | |
|---|---|---|---|---|---|
| Category | 0 | 0.5 | 1.0 | 1.5 | 2.0 |
| Bone Marrow Blasts (%) | <5 | 5 to 10 | — | 11 to 20 | 21 to 30 |
| Karyotype | Normal, | All other | Abnormal chromosome | | |
| | Y-, 5q-, 20q- | cytogenetic | 7 or 3 or more | | |
| | | abnormalities | abnormalities | | |
| Cytopenias defined as: | No cytopenia | Cytopenia of 2 | | | |
| Hemoglobin <10 g/dL | or cytopenia | or 3 cell types | | | |
| absolute neutrophil count | of 1 cell type | | | | |
| <1800/μL platelet | | | | | |
| count <100,000 μL | | | | | |

Subjects with higher-risk MDS are classified into the Revised International Prognostic Scoring System (IPSS-R) categories of Intermediate, High and Very High. This patient population largely corresponds to IPSS Intermediate risk-2 and High groups and World Health Organization (WHO) histologic subtypes of refractory anemia with excess blasts (RAEB)-1 and RAEB-2. The IPSS-R is now also considered a well-validated assessment tool to identify patients who are commonly considered clinically appropriate to receive active treatment. The Revised International Prognostic Scoring System (IPSS-R) is shown in Table 2 and includes a refined classification of cytogenetic abnormalities, more specific cut-offs for bone marrow blast counts and cytopenias, and is weighted for their severity. The Revised International Prognostic Scoring System risk groups for myelodysplastic syndromes are defined based an overall score. The overall score is calculated as the sum of the blast score, cytogenetics score, hemoglobin score, platelets score, and absolute neutrophil count score.

TABLE 2
Revised International Prognostic Scoring System (IPSS-R) Criteria and Scoring for MDS

| | | | Risk Groups |
|---|---|---|---|
| | | | (Overall Score¹ and |
| Parameter | Criteria | Score | Median Survival Time) |
| Blasts in bone marrow (%) | ≤2 | 0 | Very Low: Overall Risk Score ≤1.5 |
| | >2 to <5 | 1.0 | Median survival time = 8.8 years |
| | 5 to 10 | 2.0 | Low: Overall Risk Score >1.5 to 3.0 |
| | >10 | 3.0 | Median survival time = 5.3 years |
| Cytogenics | Very good | 0 | Intermediate: Overall Risk Score >3.0 to 4.5 |
| | Good | 1.0 | Median survival time = 3 years |
| | Intermediate | 2.0 | High: Overall Risk Score >4.5 to 6.0 |
| | Poor | 3.0 | Median survival time = 1.6 years |
| | Very Poor | 4.0 | Very High: Overall Risk Score >6.0 |
| Hemoglobin (g/dL) | ≥10 | 0 | Median survival time = 0.8 years |
| | 8 to <10 | 1.0 | |
| | <8 | 1.5 | |
| Platelets (10⁹/L) | ≥100 | 0 | |
| | 50 to <100 | 0.5 | |
| | <50 | 1.0 | |
| ANC (10⁹/L) | ≥0.8 | 0 | |
| | <8 | 0.5 | |

¹Overall score is calculated as the blast score + cytogenetics score + hemoglobin score + platelets score + absolute neutrophil count score

ECOG performance status was assessed using the criteria in Table 3.

TABLE 3
ECOG performance status

| Grade | Description |
|---|---|
| 0 | Fully active, able to carry on all pre-disease performance without |
| | restriction. |
| 1 | Restricted in physically strenuous activity but ambulatory and able to |
| | carry out work of a light or sedentary nature, e.g., light housework, |
| | office work. |
| 2 | Ambulatory and capable of all self-care but unable to carry out any |
| | work activities. Up and about more than 50% of waking hours. |
| 3 | Capable of only limited self-care, confined to bed or chair more than |
| | 50% of waking hours. |
| 4 | Completely disabled. Cannot carry on any self-care. Totally confined |
| | to bed or chair. |

### Key Exclusion Criteria

1. Subject has received prior therapy for MDS.

2. Subject has received prior therapy with a BH3 mimetic

3. Subject has a diagnosis other than previously untreated de novo MDS, including:
a. MDS with IPSS risk categories Low or Int-1 (overall IPSS score <1.5)
b. Therapy-related MDS (t-MDS)
c. MDS evolving from a pre-existing myeloproliferative neoplasm (MPN)
d. MDS/MPN including chronic myelomonocytic leukemia (CMML), atypical chronic myeloid leukemia (CIVIL), juvenile myelomonocytic leukemia (JMML) and unclassifiable MDS/MPN.

4. Subject has received strong or moderate CYP3A inducers within 7 days prior to the first dose of study drug.

5. Subject enrolled in a Dose-Escalation cohort has received strong or moderate CYP3A inhibitors within 3 days prior to the first dose of study drug with the exception of Safety Expansion cohorts.

Azacitidine 75 mg/m² (intravenous or subcutaneous daily) was administered for 7 days and venetoclax was administered at 400 mg for 14 days in each 28 day cycle. In both cohorts, dose modification during Cycle 1 was not recommended. Dose modifications in subsequent cycles were prescribed for adverse events. In Safety Expansion Cohort 1 (SE1), venetoclax was initially reduced for significant neutrophil or platelet toxicity. Dose reductions per protocol were 33% for azacitidine or 50% for venetoclax for 14 days in each cycle. In subsequent cycles, venetoclax duration could be shortened to 9 days of each cycle. In Safety Expansion Cohort 2 (SE2), dose modification guidelines recommended stepwise reductions, first in azacitidine dose (first to 50 mg/m², then 36 mg/m²) and subsequently in venetoclax duration to 7 days of each cycle (venetoclax 400 mg) as shown in Table 4. The impact of each dose modification strategy on safety and efficacy in SE1 versus SE2 was compared. Worsening of treatment-emergent adverse events grades from baseline was analyzed by cycle. Responses were evaluated using IWG 2006 criteria. Analyses included all subject who received ≥1 dose of study drug.

TABLE 4
SE2 Dose Modification Guidelines

| | Azacitidine | Venetoclax |
|---|---|---|
| Planned Dose | 75 mg/m² × 7 days | 400 mg × 14 days |
| 1^{st} Dose Reduction | 50 mg/m² × 7 days | 400 mg × 14 days |
| 2^{nd} Dose Reduction | 36 mg/m² × 7 days | 400 mg × 14 days |
| 3^{rd} Dose Reduction | 36 mg/m² × 7 days | 400 mg × 7 days |

After a previous interruption of treatment, delay starting the next cycle, onset of adverse events associated with hematological toxicity, significant reduction of neutrophils, or significant reduction of platelets, a treatment dose reduction may be indicated. Stepwise azacitidine dose modification occurred followed by adjustment of the venetoclax treatment from 14 to 7 days at the last step after all azacitidine dose modification steps had occurred. Venetoclax and azacitidine were resumed on the same day after any delays or interruptions in treatment.

Subjects who initiate a cycle of treatment with neutropenia, absolute neutrophil count <1.5×10⁹/L, or thrombocytopenia, platelets <75×10⁹/L, may be especially sensitive to cytopenias due to abnormal hematopoiesis resulting from the underlying absolute neutrophil count. Accordingly, such subjects do not typically require dose reductions in response to uncomplicated nadir cytopenias. However, subjects who initiate a cycle of treatment with absolute neutrophil count 1.5×10⁹/L and platelets 75×10⁹/L and have a previous response of complete remission, partial remission, or marrow complete remission may require dose reduction, if subsequent absolute neutrophil count nadir <0.500×10⁹/L, or platelets nadir <50×10⁹/L, if baseline was >100×10⁹/L, or platelets <50% if baseline was ≤100×10⁹/L.

Results

Baseline characteristics for SE1 and SE2 are shown in Table 5. 22 Subjects in SE1 and 21 subjects in S2 were compared with median (range) follow-up of 7.5 (1.0-8.9) and 7.9 (1.8-10.1) months, respectively, as shown in Table 6.

TABLE 5
SE1 and SE2 Baseline Characteristics

| | | |
|---|---|---|
| | SE1 | SE2 |
| n (%) | n = 22 | n = 21 |

| | | | | |
|---|---|---|---|---|
| Male | 15 | (68) | 13 | (62) |

| | | |
|---|---|---|
| Median age, years | 70 | 68 |
| [range] | [55-79] | [59-87] |

ECOG performance score

| | | | | |
|---|---|---|---|---|
| 0 | 7 | (32) | 11 | (52) |
| 1 | 13 | (59) | 9 | (43) |
| 2 | 2 | (9) | 1 | (5) |

Bone marrow blasts

| | | | | |
|---|---|---|---|---|
| ≤5% | 1 | (4.5) | 2 | (9.5) |
| >5% to ≤10% | 7 | (32) | 6 | (29) |
| >10% to ≤20% | 14 | (64) | 13 | (62) |

Baseline transfusion dependence

| | | | | |
|---|---|---|---|---|
| RBC or platelet | 13 | (59) | 10 | (48) |
| RBC | 13 | (59) | 9 | (43) |
| Platelet | 3 | (14) | 2 | (10) |

IPSS risk classification

| | | | | |
|---|---|---|---|---|
| Intermediate-2 | 17 | (77) | 15 | (71) |
| High | 5 | (23) | 6 | (29) |

IPSS-R risk classification

| | | | | |
|---|---|---|---|---|
| Intermediate | 5 | (23) | 3 | (14) |
| High | 5 | (23) | 7 | (33) |
| Very high | 12 | (54) | 11 | (52) |

Baseline cytopenias (Grade ≥3)

| | | | | |
|---|---|---|---|---|
| Neutropenia | 14 | (64) | 14 | (67) |
| Thrombocytopenia | 6 | (27) | 7 | (33) |
| Leukopenia | 10 | (46) | 10 | (48) |
| Anemia | 3 | (14) | 3 | (14) |

TABLE 6
Follow-up Time and Dose Administration

| | SE1 | SE2 |
|---|---|---|
| | n = 22 | n = 21 |
| Median follow-up, months (min, max) | 7.5 (1.0, 8.9) | 7.9 (1.8, 10.1) |

Azacitidine

| | | |
|---|---|---|
| Median duration, days | 156 | 184 |
| Cycles, median (range) | 4 (1-17) | 5 (1-11) |

Patients with dose reductions in Azacitidine, n (%):

| | | |
|---|---|---|
| 0 reductions | 16 (73) | 14 (67) |
| 1 reduction | 5 (23) | 5 (24) |
| 2 reductions | 0 | 2 (10) |
| >2 reductions | 1 (4.5) | 0 |
| Median days to dose modification | 125.5 | 115 |

Venetoclax

| | | |
|---|---|---|
| Median duration, days | 156 | 184 |
| Median days dosed | 56 | 70 |
| # of reductions in Venetoclax duration* | 3 (14) | 0 |
| Patients with ≥1 Ven dose amount reduction, n (%) | 10 (45) | 10 (48) |
| Median days to duration reduction | 52 | n/a |

*Reductions due to hematologic toxicity

The summary of adverse events in >20% of subjects is shown in Table 7. A similar frequency of ≥Grade 3 hematologic treatment-emergent adverse events (approximate %) were reported in SE1 and SE2, respectively, including anemia (14% and 33%), febrile neutropenia (46% and 48%), leukopenia (36% and 19%), neutropenia (55% and 48%) and thrombocytopenia (32% and 38%). Infections (59% and 38%) and leukopenia (36% and 19%) were more frequent in SE1 than SE2.

TABLE 7
Summary of Adverse Events

| | | |
|---|---|---|
| | SE1 | SE2 |
| n (%) | n = 22 | n = 21 |

| | | | | |
|---|---|---|---|---|
| Any Adverse Event | | | | |
| Infections | 13 | (59) | 8 | (38) |
| Neutropenia | 12 | (55) | 10 | (48) |
| Febrile neutropenia | 10 | (46) | 10 | (48) |
| Thrombocytopenia | 8 | (36) | 10 | (48) |
| Leukopenia | 8 | (36) | 4 | (19) |
| Anemia | 3 | (14) | 10 | (48) |
| Nausea | 7 | (32) | 13 | (62) |
| Constipation | 8 | (36) | 9 | (43) |
| Vomiting | 8 | (36) | 11 | (52) |
| Diarrhea | 6 | (27) | 12 | (57) |
| Grade ≥3 Adverse Events | | | | |
| Neutropenia | 12 | (55) | 10 | (48) |
| Febrile neutropenia | 10 | (46) | 10 | (48) |
| Infections | 9 | (41) | 4 | (19) |
| Leukopenia | 8 | (36) | 4 | (19) |
| Thrombocytopenia | 7 | (32) | 8 | (38) |
| Anemia | 3 | (14) | 7 | (33) |
| Serious Adverse Events^{a} | | | | |
| Febrile neutropenia | 8 | (36) | 9 | (43) |
| Infections | 8 | (36) | 3 | (14) |
| GI disorders | 3 | (13) | 3 | (14) |

^{a}Includes death, life-threatening, requiring hospitalization or surgical intervention, persistent/significant disability.
^{b}SE1: abdominal pain, diverticular perforation, and gastroesophageal reflux disease; SE2: nausea, pancreatitis, vomiting, and gastrointestinal hemorrhage

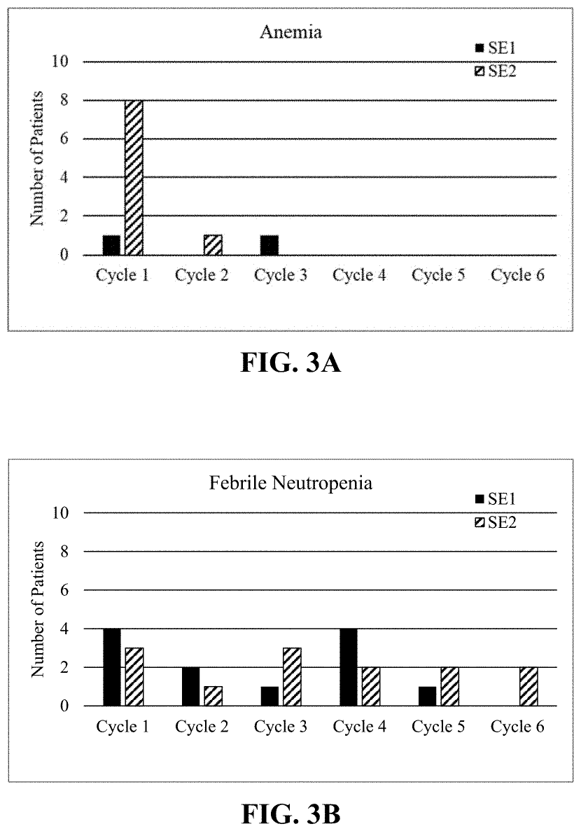

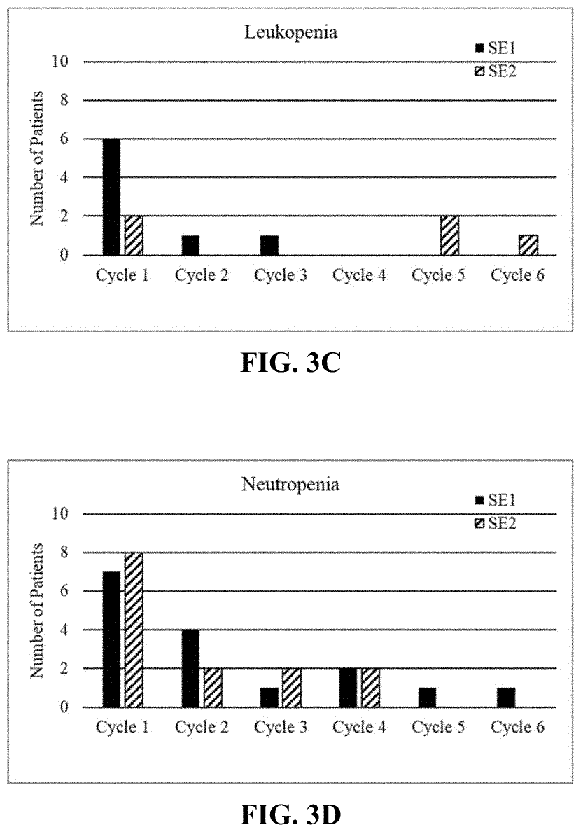

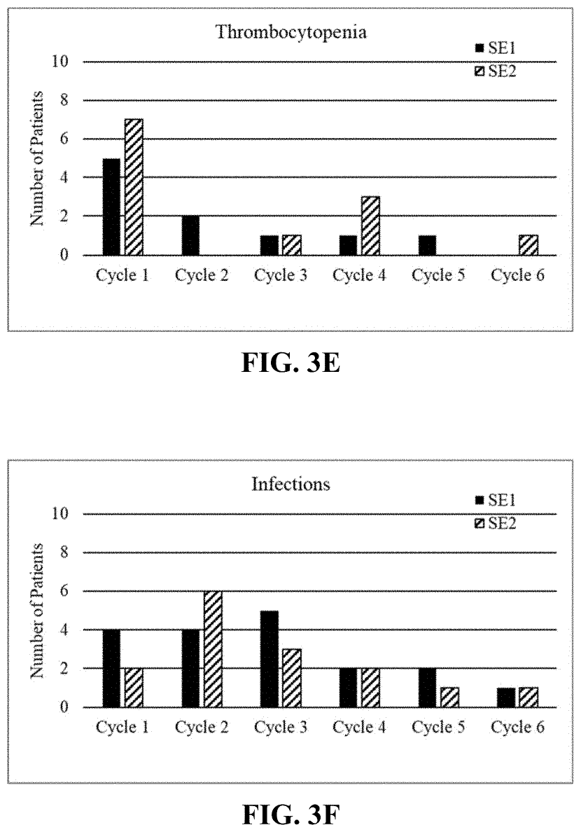

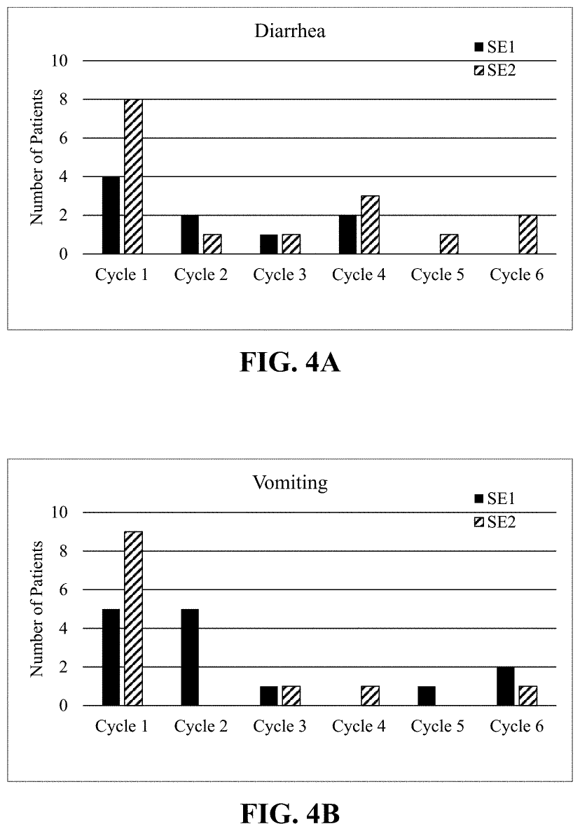

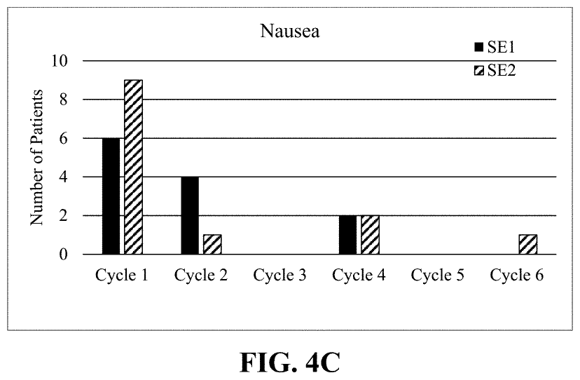

Worsening of treatment-emergent adverse events grades from baseline was analyzed by cycle. As shown in FIGS. 3A-3F and FIGS. 4A-4C, adverse event progression remains low after the first few cycles, such as cycles 1 and 2. FIGS. 3A-3F are hematologic toxicity showing the number of patients with worsening common terminology criteria grade over baseline per cycle. FIGS. 4A-4C are gastrointestinal toxicity showing the number of patients with worsening common terminology criteria grade over baseline per cycle.

Response rates were identical for SE1 and SE2 as shown in Table 8: 86% of subjects in both SE1 and SE2 had complete remission (CR) or marrow complete remission (mCR). For subjects with mCR, hematologic improvement occurred in 50% of SE1 and 46% of SE2 subjects.

TABLE 8
Response Rates

| | | |
|---|---|---|
| | SE1 | SE2 |
| n (%) | n = 22 | n = 21 |

| | | |
|---|---|---|
| Overall Response Rate | 86.30% | 85.7% |
| (CR + mCR + PR) | | |
| Best Response | | |

| | | | | |
|---|---|---|---|---|
| Complete remission | 7 | (32) | 5 | (24) |
| Marrow complete remission | 12 | (55) | 13 | (62) |
| + hematologic improvement | 6 | (50) | 6 | (46) |
| − hematologic improvement | 6 | (50) | 7 | (54) |

| | | |
|---|---|---|
| Partial remission | 0 | 0 |

| | | | | |
|---|---|---|---|---|
| Stable disease | 2 | (9) | 2 | (10) |

| | | |
|---|---|---|
| Progressive disease | 0 | 0 |

| | | | | |
|---|---|---|---|---|
| Not evaluable | 1 | (5) | 1 | (5) |

| | | |
|---|---|---|
| Time on study, median months | 7.5 | 7.9 |
| [min, max] | [1.0, 8.9] | [1.8, 10.1] |

The summary of cycle delays is shown in Table 9. Cycle delays were comparable between SE1 and SE2, with a slightly longer duration for SE1 in early cycles.

TABLE 9
Summary of Cycle Delays

| | SE1 | | | | SE2 | | | |
|---|---|---|---|---|---|---|---|---|
| | n = 22 | | | | n = 21 | | | |
| Venetoclax dose delay | | | Median # | | | | Median # days | |
| by end of each cycle | n (%) | | days (range) | | n (%) | | (range) | |
| Cycle 2 | 14 | (63) | 7.5 | (1-21) | 13 | (62) | 8 | (5-21) |
| Cycle 3 | 12 | (55) | 14 | (3-28) | 10 | (48) | 8.5 | (1-48) |
| Cycle 4 | 8 | (36) | 17 | (1-54) | 6 | (28) | 9 | (6-43) |
| Cycle 5 | 7 | (32) | 14 | (3-28) | 9 | (43) | 14 | (1-97) |
| Cycle 6 | 7 | (32) | 7 | (1-21) | 5 | (24) | 14 | (6-91) |

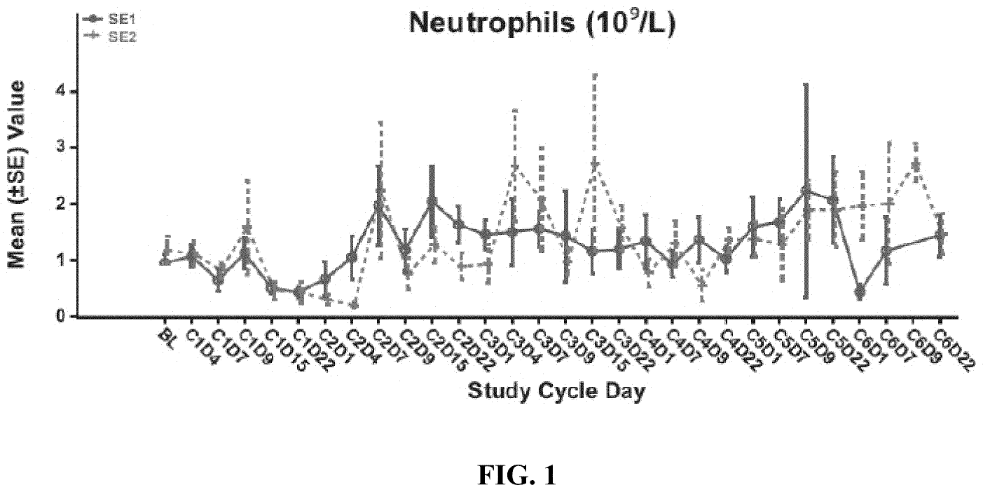

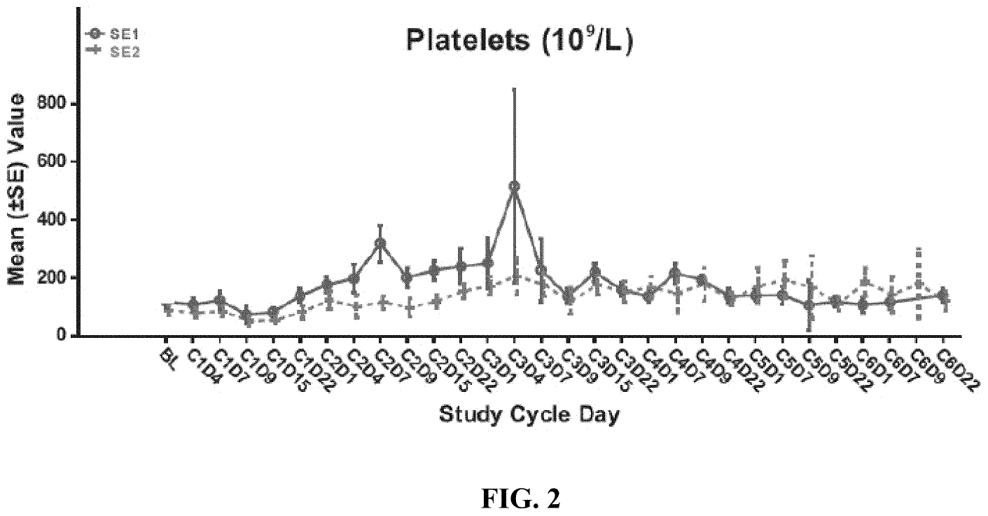

The mean value of absolute neutrophil count and platelet count is shown in FIGS. 1 and 2, respectively. The number of count observations per study cycle day for both absolute neutrophil count and platelet count is shown in Table 10.

TABLE 10
Number of Count Observations per Study Cycle Day

| | |
|---|---|
| Absolute Neutrophil | Platelet Count |
| Count Observations | Observations |

| Study Cycle Day | SE1 | SE2 | SE1 | SE2 |
|---|---|---|---|---|
| Baseline | 22 | 21 | 19 | 19 |
| C1D4 | 16 | 19 | 15 | 18 |
| C1D7 | 11 | 13 | 9 | 12 |
| C1D9 | 9 | 8 | 8 | 7 |
| C1D15 | 20 | 20 | 20 | 17 |
| C1D22 | 20 | 18 | 19 | 16 |
| C2D1 | 8 | 12 | 8 | 12 |
| C2D4 | 5 | 6 | 5 | 6 |
| C2D7 | 9 | 12 | 9 | 11 |
| C2D9 | 7 | 6 | 7 | 6 |
| C2D15 | 17 | 17 | 17 | 16 |
| C2D22 | 19 | 16 | 20 | 17 |
| C3D1 | 15 | 14 | 15 | 13 |
| C3D4 | 3 | 3 | 3 | 3 |
| C3D7 | 9 | 10 | 8 | 10 |
| C3D9 | 4 | 4 | 4 | 4 |
| C3D15 | 13 | 16 | 12 | 16 |
| C3D22 | 11 | 13 | 11 | 12 |
| C4D1 | 15 | 11 | 15 | 12 |
| C4D7 | 11 | 5 | 10 | 5 |
| C4D9 | 3 | 5 | 3 | 5 |
| C4D22 | 13 | 11 | 13 | 12 |
| C5D1 | 8 | 5 | 8 | 6 |
| C5D7 | 6 | 6 | 6 | 6 |
| C5D9 | 2 | 2 | 2 | 2 |
| C5D22 | 13 | 12 | 13 | 12 |
| C6D1 | 4 | 8 | 4 | 7 |
| C6D7 | 7 | 5 | 7 | 5 |
| C6D9 | 0 | 2 | 0 | 2 |
| C6D22 | 10 | 9 | 10 | 9 |

86% of patients in both SE1 and SE2 had complete remission or marrow complete remission.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including those relating to the methods of use of the invention, may be made without departing from the spirit and scope thereof All publications, patents, and patent applications cited herein are hereby incorporated by reference in their entirety for all purposes.

We claim:

1. A method for treating myelodysplastic syndromes in a human subject, comprising administering to the human subject a daily dose of 400 mg of venetoclax for 14 days in a 28 day dosing cycle, and a daily dose of 75 mg/m² of azacitidine for 7 days in the 28 day dosing cycle;
wherein, if the human subject has:
a baseline absolute neutrophil count of <1.5×10⁹/L, a baseline white blood cell count of <3×10⁹/L, or a baseline platelet count of <75×10⁹/L;
no improvement in cell line differentiation; and
at least one of a condition selected from the group consisting of:
a. an absolute neutrophil count nadir of ≥50% reduction;
b. a white blood cell count nadir of ≥50% reduction; and
c. a platelet count nadir of ≥50% reduction;
the daily dose of azacitidine of 75 mg/m² is reduced to ≤75% but no less than 33% during a next 28 day dosing cycle.

2. The method of claim 1, wherein the myelodysplastic syndromes are treatment-naïve higher-risk myelodysplastic syndromes.

3. The method of claim 2, wherein the daily dose of azacitidine of 75 mg/m² is reduced to 75% during the next 28 day dosing cycle;
wherein the human subject has a bone marrow cellularity of 30-60%; and
wherein the human subject has at least one of a condition selected from the group consisting of:
a. an absolute neutrophil count nadir decrease of ≥75% from the baseline absolute neutrophil count;
b. a white blood cell count nadir decrease of ≥75% from the baseline white blood cell count; and
c. a platelet count nadir decrease of >75% from the baseline platelet count.

4. The method of claim 3, wherein the azacitidine is administered intravenously.

5. The method of claim 3, wherein the azacitidine is administered subcutaneously.

6. The method of claim 3, wherein the human subject has prior to a start of the next 28 day dosing cycle, at least one condition selected from the group consisting of:
a. a next absolute neutrophil count of ≤25% above the absolute neutrophil count nadir and relative to a difference of the baseline absolute neutrophil count and the absolute neutrophil count nadir;
b. a next white blood cell count of ≤25% above the white blood cell count nadir and relative to a difference of the baseline white blood cell count and the white blood cell count nadir; and
c. a next platelet count of ≤25% above the platelet count nadir and relative to a difference of the baseline platelet count and the platelet count nadir.

7. The method of claim 6, further comprising a delay prior to the next 28 day dosing cycle of >1 day.

8. The method of claim 6, wherein the daily dose of 400 mg of venetoclax is reduced from 14 to 7 days in a subsequent 28 day dosing cycle;
wherein the human subject has prior to a start of the subsequent 28 day dosing cycle, at least one condition selected from the group consisting of:
a. a subsequent absolute neutrophil count of ≤25% above the absolute neutrophil count nadir and relative to a difference of the baseline absolute neutrophil count and the absolute neutrophil count nadir;
b. a next white blood cell count of ≤25% above the white blood cell count nadir and relative to a difference of the baseline white blood cell count and the white blood cell count nadir; and
c. a next platelet count of ≤25% above the platelet count nadir and relative to a difference of the baseline platelet count and the platelet count nadir.

9. The method of claim 2, wherein the daily dose of azacitidine of 75 mg/m² is reduced to 50% during the next 28 day dosing cycle;
wherein the human subject has a bone marrow cellularity of 15 to <30%; and
wherein the human subject has at least one of a condition selected from the group consisting of:
a. an absolute neutrophil count nadir decrease of ≥50% from the baseline absolute neutrophil count;
b. a white blood cell count nadir decrease of ≥50% from the baseline white blood cell count; and
c. a platelet count nadir decrease of ≥50% from the baseline platelet count.

10. The method of claim 9, wherein the azacitidine is administered intravenously.

11. The method of claim 9, wherein the azacitidine is administered subcutaneously.

12. The method of claim 9, wherein the human subject has prior to a start of the next 28 day dosing cycle, at least one condition selected from the group consisting of:
a. a next absolute neutrophil count of ≤25% above the absolute neutrophil count nadir and relative to a difference of the baseline absolute neutrophil count and the absolute neutrophil count nadir;
b. a next white blood cell count of ≤25% above the white blood cell count nadir and relative to a difference of the baseline white blood cell count and the white blood cell count nadir; and
c. a next platelet count of ≤25% above the platelet count nadir and relative to a difference of the baseline platelet count and the platelet count nadir.

13. The method of claim 12, further comprising a delay prior to the next 28 day dosing cycle of >1 day.

14. The method of claim 12, wherein the daily dose of 400 mg of venetoclax is reduced from 14 to 7 days in a subsequent 28 day dosing cycle;
wherein the human subject has prior to a start of the subsequent 28 day dosing cycle, at least one condition selected from the group consisting of:
a. a subsequent absolute neutrophil count of ≤25% above the absolute neutrophil count nadir and relative to a difference of the baseline absolute neutrophil count and the absolute neutrophil count nadir;
b. a subsequent white blood cell count of ≤25% above the white blood cell count nadir and relative to a difference of the baseline white blood cell count and the white blood cell count nadir; and
c. a subsequent platelet count of ≤25% above the platelet count nadir and relative to a difference of the baseline platelet count and the platelet count nadir.

15. The method of claim 2, wherein the daily dose of azacitidine of 75 mg/m² is reduced to 33% during the next 28 day dosing cycle;
wherein the human subject has a bone marrow cellularity <15%; and
wherein the human subject has at least one of a condition selected from the group consisting of:
a. an absolute neutrophil count nadir decrease of ≥50% from the baseline absolute neutrophil count;
b. a white blood cell count nadir decrease of ≥50% from the baseline white blood cell count; and
c. a platelet count nadir decrease of ≥50% from the baseline platelet count.

16. The method of claim 15, wherein the azacitidine is administered intravenously.

17. The method of claim 15, wherein the azacitidine is administered subcutaneously.

18. The method of claim 15, wherein the human subject has prior to a start of the next 28 day dosing cycle, at least one condition selected from the group consisting of:
a. a next absolute neutrophil count of ≤25% above the absolute neutrophil count nadir and relative to a difference of the baseline absolute neutrophil count and the absolute neutrophil count nadir;
b. a next white blood cell count of ≤25% above the white blood cell count nadir and relative to a difference of the baseline white blood cell count and the white blood cell count nadir; and
c. a next platelet count of ≤25% above the platelet count nadir and relative to a difference of the baseline platelet count and the platelet count nadir.

19. The method of claim 18, further comprising a delay prior to the next 28 day dosing cycle of ≥1 day.

20. The method of claim 18, wherein the daily dose of 400 mg of venetoclax is reduced from 14 to 7 days in a subsequent 28 day dosing cycle;
wherein the human subject has prior to a start of the subsequent 28 day dosing cycle, at least one condition selected from the group consisting of:
a. a subsequent neutrophil count of >25% above the absolute neutrophil count nadir and relative to a difference of the baseline absolute neutrophil count and the absolute neutrophil count nadir;
b. a subsequent white blood cell count of >25% above the white blood cell count nadir and relative to a difference of the baseline white blood cell count and the white blood cell count nadir; and
c. a subsequent platelet count of >25% above the platelet count nadir and relative to a difference of the baseline platelet count and the platelet count nadir.

21. A method for treating myelodysplastic syndromes in a human subject, comprising administering to the human subject a daily dose of 400 mg of venetoclax for 14 days in a 28 day dosing cycle, and a daily dose of 75 mg/m² of azacitidine for 7 days in the 28 day dosing cycle;
wherein, if the human subject has:
a baseline absolute neutrophil count of ≥1.5×10⁹/L,
a baseline white blood cell count of ≥3×10⁹/L, or
a baseline platelet count of ≥75×10⁹/L; and
at least one of a condition selected from the group consisting of:
a. an absolute neutrophil count nadir of ≤1.5×10⁹/L; and
b. a platelet count nadir of ≤50×10⁹/L;
the daily dose of azacitidine is reduced from 75 mg/m² to ≤67% but no less than 50% during the next 28 day dosing cycle.

22. The method of claim 21, wherein the myelodysplastic syndromes are treatment-naïve higher-risk myelodysplastic syndromes.

23. The method of claim 22, wherein the daily dose of azacitidine is reduced from 75 mg/m² to 67%; and
wherein the human subject has at least one condition selected from the group consisting of:
a. the absolute neutrophil count nadir is from 0.5×10⁹/L to 1.5×10⁹/L; and
b. the platelet count nadir is from 25×10⁹/L to 50×10⁹/L.

24. The method of claim 23, wherein the human subject has prior to a start of the next 28 day dosing cycle, at least one condition selected from the group consisting of:
a. a next absolute neutrophil count of ≤1.5×10⁹/L; and
b. a next platelet count of ≤50×10⁹/L.

25. The method of claim 24, further comprising a delay prior to the next 28 day dosing cycle of >1 day.

26. The method of claim 24, wherein the daily dose of 400 mg of venetoclax is reduced from 14 to 7 days in a subsequent 28 day dosing cycle;
wherein the human subject has prior to a start of the subsequent 28 day dosing cycle at least one condition selected from the group consisting of:
a. a subsequent absolute neutrophil count of ≤1.5×10⁹/L; and
b. a subsequent platelet count of ≤50×10⁹/L.

27. The method of claim 22, wherein the daily dose of azacitidine is reduced from 75 mg/m² to 50%; and
wherein the human subject has at least one condition selected from the group consisting of:
a. the absolute neutrophil count nadir is <0.5×10⁹/L; and
b. the platelet count nadir is <25×10⁹/L.

28. The method of claim 27, wherein the human subject has prior to a start of the next 28 day dosing cycle, at least one condition selected from the group consisting of:
a. a next absolute neutrophil count of ≤1.5×10⁹/L; and
b. a next platelet count of ≤50×10⁹/L.

29. The method of claim 28, further comprising a delay prior to the next 28 day dosing cycle of ≥1 day.

30. The method of claim 28, wherein the daily dose of 400 mg of venetoclax is reduced from 14 to 7 days in a subsequent 28 day dosing cycle;
wherein the human subject has prior to a start of the subsequent 28 day dosing cycle at least one condition selected from the group consisting of:
a. a subsequent absolute neutrophil count of ≤1.5×10⁹/L; and
b. a subsequent platelet count of ≤50×10⁹/L.
